(19) United States
(12) Patent Application Publication
(10) Pub. No.: US 2010/0076570 A1
(43) Pub. Date: Mar. 25, 2010

(54) MEDICAL IMPLANT

(76) Inventors: Tim Band, Derbyshire (GB); Derek McMinn, Birmingham (GB)

Correspondence Address: DIANA HOUSTON;SMITH & NEPHEW, INC., 1450 BROOKS ROAD, MEMPHIS, TN, 38116, US

(21) Appl. No.: 12/442,780

(22) PCT Filed: Sep. 24, 2007
(86) PCT No.: PCT/GB07/03641

§ 371 Date: Apr. 22, 2009

(30) Foreign Application Priority Data
Sep. 26, 2006 (GB) 0618930.2

(51) Int. Cl. A61F 2/36; A61F 2/28; A61B 17/56

(52) U.S. Cl. 623/23.12; 623/23.6; 606/86 R

(57) ABSTRACT

The present invention provides an implant (1) comprising a hollow body (2) having an opening (8), the body (2) having an inner (3) and an outer (4) surface, wherein the inner surface (3) of the body (2) has a surface structure (9) that enables bone in-growth. The present invention also provides a method of implantation, comprising: preparing a resected bone with an optional cavity in the bone canal; providing such an implant (1); and implanting the implant.

The present invention relates to an implant, in particular a femoral implant.

Femoral implants are typically attached to a resected femur by a friction fit between the stem of the femoral implant and a cavity prepared in the medullary canal of the femur. The medullary canal is usually reamed so as to produce a cavity that is undersized with respect to the dimensions of the implant stem in order to provide a suitable friction fit. However, a friction fit alone cannot guarantee stability and the stem can work itself loose during use leading to damage to the femur and the need for surgical revision.

In order to increase the stability of the implant cement can be used to fix the stem inside the cavity formed in the medullary canal. However, the use of cement has documented drawbacks and is often not the surgeon's preferred method.

It is therefore an aim of the present invention to provide an implant with increased stability compared to conventional implants and which does not rely on the stem being fixed into the cavity by friction fit or cement alone.

According to a first aspect of the present invention, there is provided an implant comprising a hollow body having an opening, the body having an inner and an outer surface, wherein the inner surface of the body has a surface structure that enables bone in-growth.

The present invention has the advantage that the inner surface of the body provides a surface for bone in-growth, which leads to similar stability as conventional implants, without the need for the use of cement to fix the body to the bone.

According to embodiments of the present invention, the inner surface of the body is shaped so as to enable bone in-growth. This means that the inner surface has a surface structure (texture) that allows bone in-growth. The surface structure may be configured so that it promotes bone in-growth. Preferably, the surface structure is at least in part porous.

The surface structure may be coated with a material. The material may stimulate bone in-growth. For example, the surface structure may comprise a hydroxyapatite (HA) coating, or the like.

The inner surface may comprise projections. The inner surface may be at least in part covered with projections.

The projections may be coated with a material. The material may stimulate bone in-growth. For example, the projections may comprise a hydroxyapatite (HA) coating, or the like.

The majority of the inner surface may be free of projections.

The inner surface may be at least in part covered with recesses. The inner surface may comprise a combination of recesses and projections.

The majority of the inner surface may be covered with projections. Substantially all of the inner surface may be covered with projections.

Projections that are immediately adjacent to each other may be totally separated from one another.

Projections that are immediately adjacent to each other may be in contact with each other, provided that there is at least some separation between projections to allow bone in-growth.

Projections that are immediately adjacent to each other may be in contact with at least one adjacent projection and separated from at least one adjacent projection.

The projections on the inner surface of the body may have any suitable shape to enable bone in-growth. For example, the projections may have regular geometrical shapes such as n-sided blocks wherein n is greater than two (particularly square blocks, oblong blocks, pentagonal blocks, hexagonal blocks, pyramids and such like), cylinders, cones, partial spheres (for example hemispheres) or any combination of such shapes.

The projections may have an amorphous shape.

Preferably, the projections on the inner surface of the body are at least in part in the form of beads. The beads may have a diameter in the range 0.05-2.0 mm. The beads may have a diameter in the range 0.1-1.5 mm. The beads may have a diameter in the range 0.1-1.0 mm. Preferably, the average bead diameter is around 0.25-0.5 mm.

The height that the beads project from the inner surface of the body may be in the range 0.05-2.0 mm. The height that the beads project from the inner surface of the body may be in the range 0.1-1.5 mm. The height that the beads project from the inner surface of the body may be in the range 0.1-1.0 mm. Preferably, the average height that the beads project from the inner surface of the body is around 0.25-0.5 mm.

Beads that are immediately adjacent to each other may be totally separated from one another.

Beads that are immediately adjacent to each other may be in contact with each other, provided that there is at least some separation between projections to allow bone in-growth.

Beads that are immediately adjacent each other may be in contact with at least one adjacent bead and separated from at least one adjacent bead.

The separation between immediately adjacent beads may be in the range 0.05-2.0 mm. The bead separation may be in the range 0.1-1.5 mm. The bead separation may be in the range 0.1-1.0 mm. Preferably, the average bead separation is 0.15-0.45 mm.

The beads may form a single layer. The beads may form a plurality of layers. For example, there may be two, three, four, five or more layers of beads.

The beads may be integrally cast with the body. Any conventional casting technique may be used for example vacuum or air casting. Preferably, the Porocast™ process is used to form a cast-in porous surface in which the beads are integral with the body.

Alternatively, the beads may be formed on the inner surface after casting of the body. The beads may be formed post-casting by plasma spray, adhesive bonded spray, low temperature sintering or high temperature sintering.

The inner surface of the body may have a surface structure that mimics trabecular bone. For example, the inner surface may be trabecular metal. Trabecular metal comprises interconnecting pores that enable bone in-growth.

The implant body may be at least in part curved. The outer surface of the body may be at least in part convex. The inner surface of the body may be at least in part concave. The inner surface may comprise one or more flat or substantially flat portions.

The body may be part-spherical. The body may be at least in part hemispherical. The body may be more than hemispherical. The body may encompass 50-75% of a sphere.

In those embodiments in which the implant body is part spherical, the body may have a diameter in the range 20-75 mm. The body may have a diameter in the range 25-70 mm. The body may have a diameter in the range 25-65 mm. Preferably, the diameter of the body is in the range 35-65 mm.

The depth of the implant body may be in the range 15-70 mm. The depth may be in the range 20-60 mm. Preferably, the depth is in the range 20-50 mm.

The thickness of the body measured between the inner and outer surfaces may be in the range 0.75-20 mm.

The opening of the body may be circular or substantially circular. The circular opening may have a diameter in the range 15-70 mm.

The implant body may be made from plastic, metal or ceramic. Preferably, the implant body is made from metal. The metal may be stainless steel. The metal may be titanium. The metal may be an alloy. The alloy may be cobalt-chrome alloy. The metal, metal alloy or ceramic could be coated or surface modified. The surface modification could be ceramic bonded to metal, oxinium, niobium nitride or titanium nitride.

According to preferred embodiments of the present invention, the implant may further comprise a stem attached to the inner surface of the body.

The stem may extend through the opening in the body.

The stem may be fixedly attached to the body. The stem may be integrally cast with the body.

The stem may be removably attached to the body. The body and stem may have corresponding threads.

The main axis of the stem may be co-linear with the centre of the opening in the body.

The stem may have a length in the range 10-200 mm.

The stem may be cylindrical. The stem may have a diameter in the range 3-15 mm.

The stem may be tapered. The proximal stem diameter may range from 3-40 mm. The distal stem diameter may range from 2-35 mm.

The implant stem may be made from plastic, metal, ceramic or a resorbable material. Preferably, the implant stem is made from metal. The metal may be stainless steel. The metal may be titanium. The metal may be an alloy.

In use, the implant stem may be attached to the bone by a press fit between the bone cavity and the stem.

In use, cement may be used to attach the implant stem to the bone.

The implant may be a femoral implant.

According to a second aspect of the present invention, there is provided a method of implantation, comprising:
preparing a resected bone with an optional cavity in the bone canal;
providing an implant according to the first aspect;
implanting the implant.

The bone may be a femur.

Reference will now be made, by way of example, to the accompanying drawings in which:

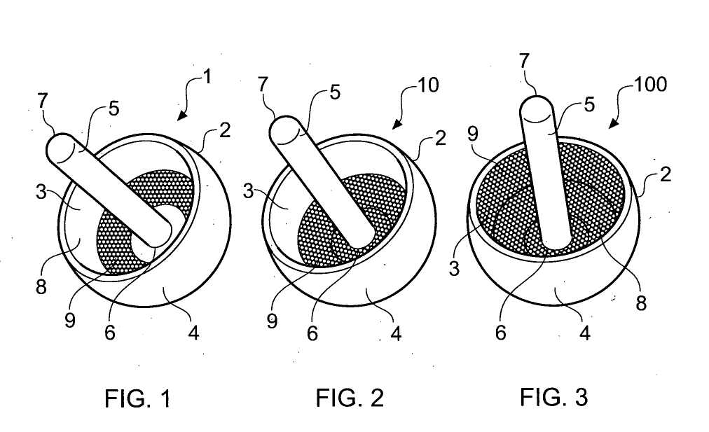

FIG. 1 shows a perspective view of an implant according to an embodiment of the present invention;

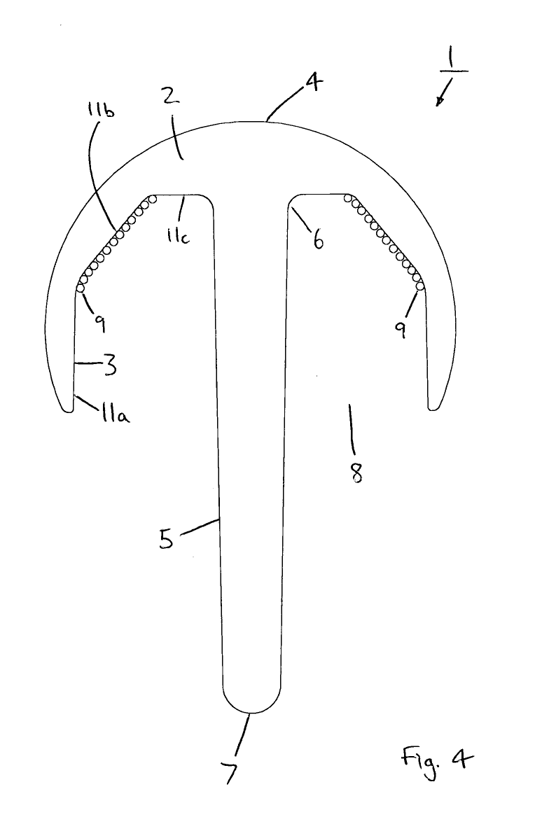

FIG. 2 shows a perspective view of an implant according to another embodiment of the present invention;

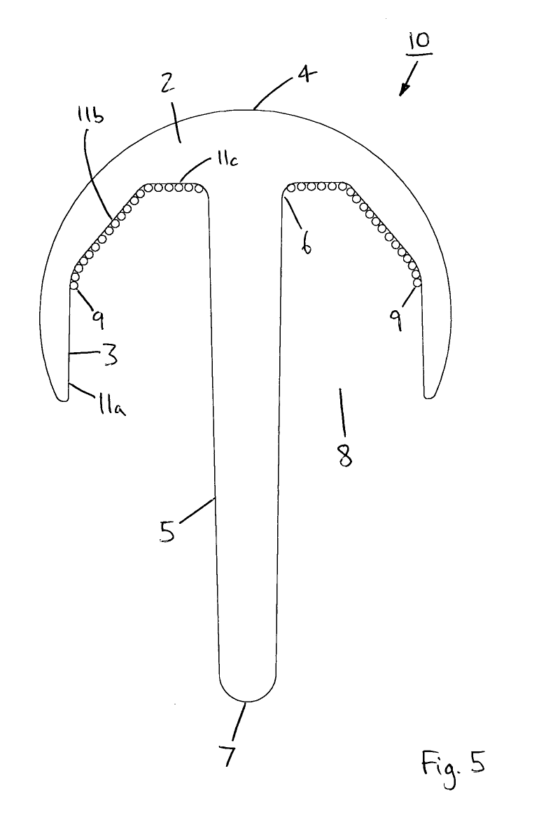

FIG. 3 shows a perspective view of an implant according to another embodiment of the present invention;

FIG. 4 shows a cross-section of the implant of FIG. 1;

FIG. 5 shows a cross-section of the implant of FIG. 2;

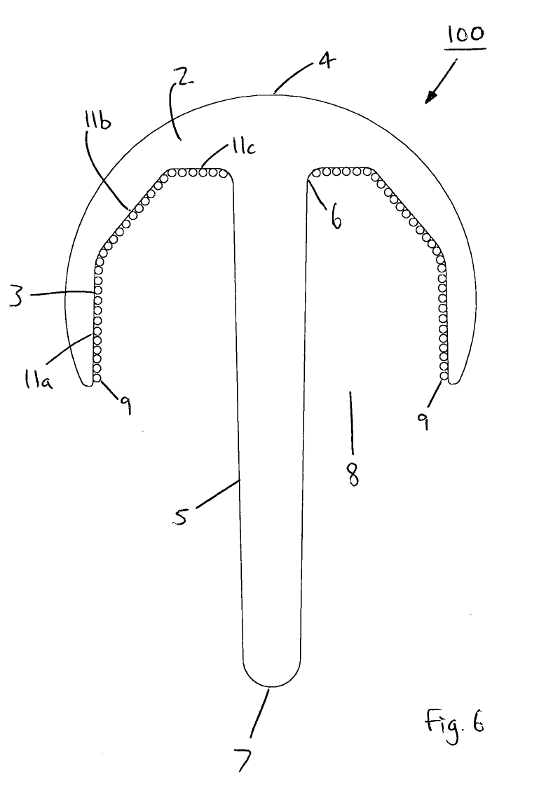

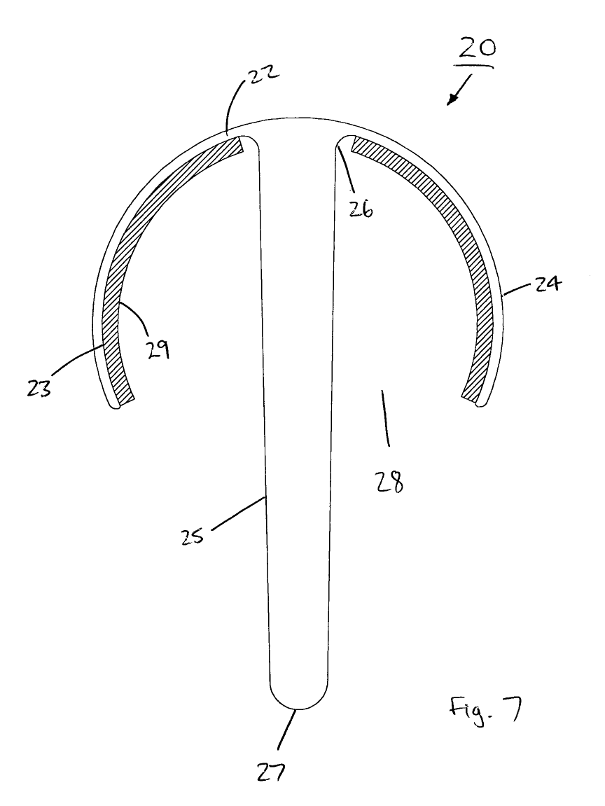

FIG. 6 shows a cross-section of the implant of FIG. 3;

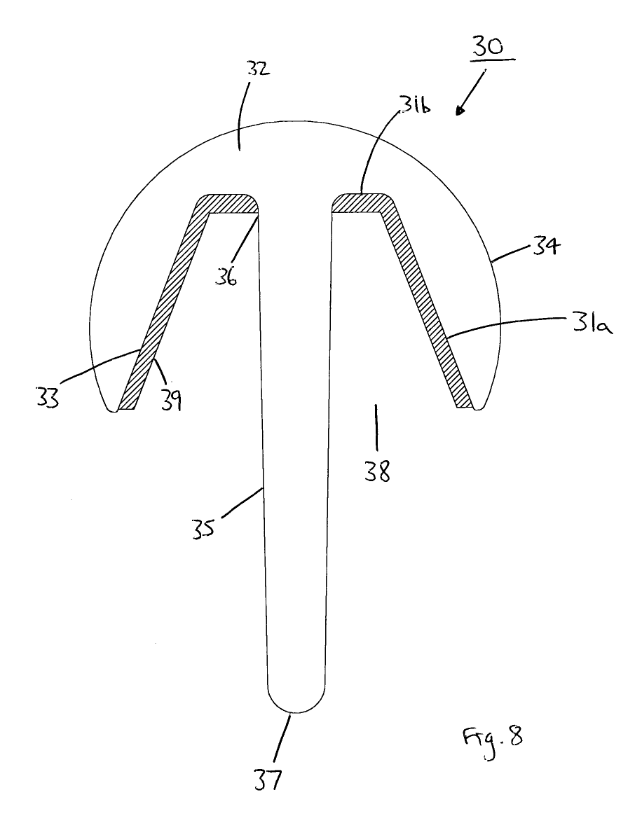

FIG. 7 shows a cross-section of an implant according to another embodiment of the present invention;

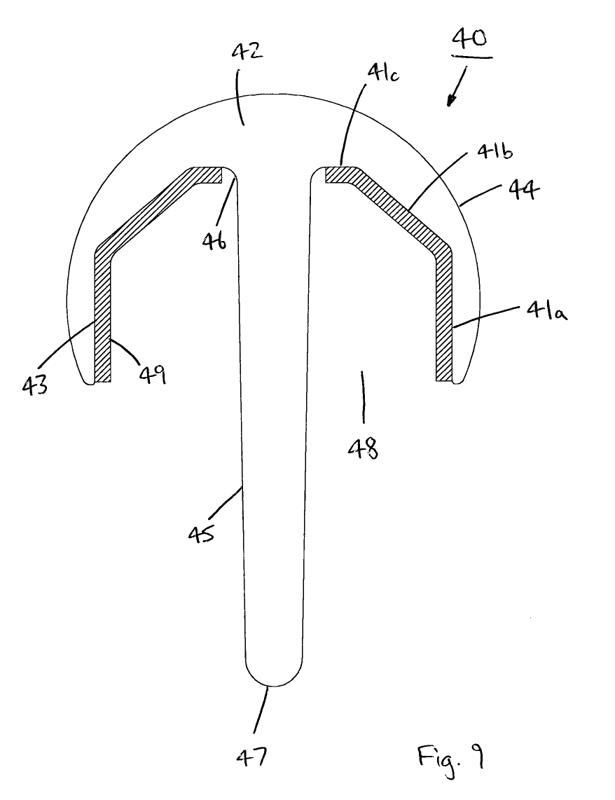

FIG. 8 shows a cross-section of an implant according to another embodiment of the present invention;

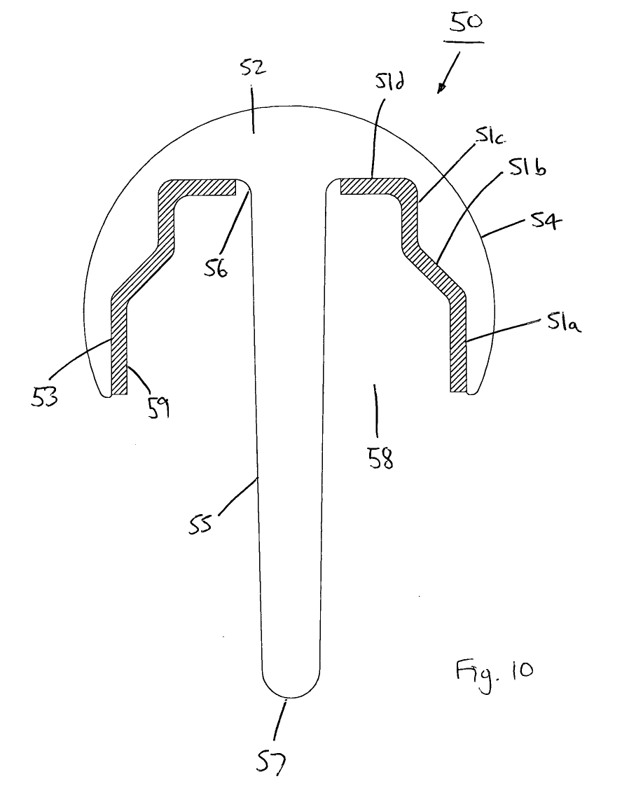

FIG. 9 shows a cross-section of an implant according to another embodiment of the present invention;

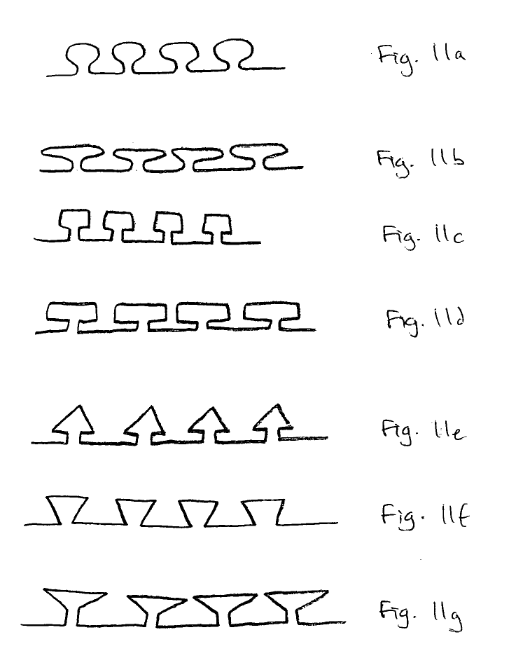

FIG. 10 shows a cross-section of an implant according to another embodiment of the present invention; and

FIGS. 11a-g show cross-sections of projections according to embodiments of the present invention.

As shown in FIG. 1, the implant body (2) is based on a sphere with the outer surface (4) of the body (2) forming a shape that is more than hemispherical. The implant (1) comprises a stem (5) having a first end (6) and a second end (7). The first end (6) of the stem (5) is fixed to the inner surface (3) of the pole of the implant body (2), such that the main axis of the stem (5) is coaxial with the opening (8) in the body (2). The second end (7) of the stem (5) extends through the opening (8) in the body (2). The inner surface (3) of the implant body (2) comprises a plurality of beads (9) that partially cover the inner surface (3) of the implant body (2) in a concentric band. There are no beads (9) in the proximity of the first end (6) of the stem (5) and no beads (9) towards the opening (8) of the implant body (2). The band of beads (9) covers around 25-35% of the inner surface (3) of the body (2).

The implant body (2) may have a diameter in the range 20-75 mm. The body (2) may have a diameter in the range 25-70 mm. The body (2) may have a diameter in the range 25-65 mm. Preferably, the diameter of the body (2) is in the range 35-65 mm.

The depth of the implant body (2) may be in the range 15-70 mm. The depth may be in the range 20-60 mm. Preferably, the depth is in the range 20-50 mm.

The thickness of the body (2) measured between the inner (3) and outer (4) surfaces may be in the range 0.75-20 mm.

The opening (8) of the body (2) may be circular or substantially circular. The circular opening (8) may have a diameter in the range 15-70 mm.

The implant body (2) may be made from plastic, metal or ceramic. Preferably, the implant body (2) is made from metal. The metal may be stainless steel. The metal may be titanium. The metal may be an alloy. The alloy may be cobalt-chrome alloy. The metal, metal alloy or ceramic could be coated or surface modified. The surface modification could be ceramic bonded to metal, oxinium, niobium nitride or titanium nitride, or carburisation.

The implant stem (5) may have a length in the range 10-200 mm. The stem (5) may have a diameter in the range 3-15 mm.

The implant stem (5) may be made from plastic, metal or ceramic. Preferably, the implant stem (5) is made from metal. The metal may be stainless steel.

The metal may be titanium. The metal may be an alloy. The stem (5) may be integrally cast with the body (2).

The beads (9) may have a diameter in the range 0.05-2.0 mm. The beads (9) may have a diameter in the range 0.1-1.5 mm. The beads (9) may have a diameter in the range 0.1-1.0 mm. Preferably, the average bead diameter is around 0.25-0.5 mm.

The height that the beads (9) project from the inner surface (3) of the body (2) may be in the range 0.05-2.0 mm. The height that the beads (9) project from the inner surface (3) of the body (2) may be in the range 0.1-1.5 mm. The height that the beads (9) project from the inner surface (3) of the body (2) may be in the range 0.1-1.0 mm. Preferably, the average height that the beads (9) project from the inner surface (3) of the body (2) is around 0.25-0.5 mm.

Beads (9) that are immediately adjacent to each other may be totally separated from one another.

Beads (9) that are immediately adjacent to each other may be in contact with each other, provided that there is at least some separation between beads to allow bone in-growth.

Beads (9) that are immediately adjacent each other may be in contact with at least one adjacent bead (9) and separated from at least one adjacent bead (9).

The separation between immediately adjacent beads (9) may be in the range 0.05-2.0 mm. The bead separation may be in the range 0.1-1.5 mm. The bead separation may be in the range 0.1-1.0 mm. Preferably, the average bead separation is 0.15-0.45 mm.

The beads (9) may be integrally cast with the body (2). Any conventional casting technique may be used for example vacuum or air casting.

Preferably, the Porocast™ process is used to form a cast-in porous surface in which the beads (9) are integral with the body (2).

Alternatively, the beads (9) may be formed on the inner surface (3) after casting of the body (2). The beads (9) may be formed post-casting by plasma spray, adhesive bonded spray, low temperature sintering or high temperature sintering.

FIG. 2 shows an implant (10) according to another embodiment of the present invention. The structure and dimensions of the implant body (2), the implant stem (5) and the beads (9) are the same as those shown in FIG. 1. However, the inner surface (3) has a plurality of beads (9) that partially cover the inner surface (3) of the implant body (2) to a greater extent than that of the embodiment of FIG. 1. As shown, the beads (9) extend from the proximity of the first end (6) of the stem (5) to around the mid-point of the implant body (2). The band of beads (9) covers around 40-60% of the inner surface (3).

FIG. 3 shows an implant (100) according to another embodiment of the present invention. The structure and dimensions of the implant body (2), the implant stem (5) and the beads (9) are the same as that shown in FIG. 1. However, the inner surface (3) has a plurality of beads (9) that cover substantially all of the inner surface (3) of the implant head (2).

FIGS. 4 to 6 show cross-sections of the implants (1, 10, 100) of FIGS. 1 to 3, respectively. The porous surface structure provided by the beads (9) can be seen. The inner surface (3) comprises three substantially flat sections (11a,b,c). The outer surface (4) is curved. In FIG. 4, section 11b of the inner surface (3) has a surface structure comprising beads (9). In FIG. 5, sections 11b and 11c of the inner surface (3) have a surface structure comprising beads (9). In FIG. 6, sections 11a, 11b, and 11c of the inner surface (3) have a surface structure comprising beads (9).

FIG. 7 shows a cross-section of an implant (20) according to another embodiment of the present invention. The implant body (22) is based on a sphere with the inner (23) and outer (24) surfaces of the body (22) forming a shape that is more than hemispherical. The implant (20) comprises a stem (25) having a first end (26) and a second end (27). The first end (26) of the stem (25) is fixed to the inner surface (23) of the pole of the implant body (22), such that the main axis of the stem (25) is coaxial with the opening (28) in the body (22). The second end (27) of the stem (25) extends through the opening (28) in the body (22).

The inner surface (23) of the implant body (22) comprises a surface structure (29) that enables bone in-growth. The surface structure (29) is shown as a generic feature for the purpose of clarity. The surface structure (29) is shown covering substantially all of the inner surface (23). However, the surface structure (29) may partially cover the inner surface (23), as described earlier in relation to FIGS. 1, 2, 4 and 5. The surface structure (29) may comprise projections as described herein (see for example FIGS. 11a-g). The surface structure (29) may comprise beads as described herein (see for example FIGS. 1 to 6). The surface structure (29) may mimic trabecular bone. For example, the surface structure (29) may be trabecular metal.

The implant body (22) may have a diameter in the range 20-75 mm. The body (22) may have a diameter in the range 25-70 mm. The body (22) may have a diameter in the range 25-65 mm. Preferably, the diameter of the body (22) is in the range 35-65 mm.

The depth of the implant body (22) may be in the range 15-70 mm. The depth may be in the range 20-60 mm. Preferably, the depth is in the range 20-50 mm.

The thickness of the body (22) measured between the inner (23) and outer (24) surfaces may be in the range 0.75-20 mm.

The opening (28) of the body (22) may be circular or substantially circular. The circular opening (28) may have a diameter in the range 15-70 mm.

The implant body (22) may be made from plastic, metal or ceramic. Preferably, the implant body (22) is made from metal. The metal may be stainless steel. The metal may be titanium. The metal may be an alloy. The alloy may be cobalt-chrome alloy. The metal, metal alloy or ceramic could be coated or surface modified. The surface modification could be ceramic bonded to metal, oxinium, niobium nitride or titanium nitride, or carburisation.

The implant stem (25) may have a length in the range 10-200 mm. The stem (25) may have a diameter in the range 3-15 mm.

The implant stem (25) may be made from plastic, metal or ceramic. Preferably, the implant stem (25) is made from metal. The metal may be stainless steel. The metal may be titanium. The metal may be an alloy. The stem (25) may be integrally cast with the body (22).

FIG. 8 shows a cross-section of an implant (30) according to another embodiment of the present invention. The implant body (32) is based on a sphere with the outer surface (34) of the body (32) forming a shape that is more than hemispherical. The inner surface (33) comprises two substantially flat sections (31a,b). The implant (30) comprises a stem (35) having a first end (36) and a second end (37). The first end (36) of the stem (35) is fixed to the inner surface (33) of the pole of the implant body (32), such that the main axis of the stem (35) is coaxial with the opening (38) in the body (32). The second end (37) of the stem (35) extends through the opening (38) in the body (32).

The inner surface (33) of the implant body (32) comprises a surface structure (39) that enables bone in-growth. The surface structure (39) is shown as a generic feature for the purpose of clarity. The surface structure (39) is shown covering substantially all of the inner surface (33). However, the surface structure (39) may partially cover the inner surface (33), as described earlier in relation to FIGS. 1, 2, 4 and 5. The surface structure (39) may comprise projections as described herein (see for example FIGS. 11a-g). The surface structure (39) may comprise beads as described herein (see for example FIGS. 1 to 6). The surface structure (39) may mimic trabecular bone. For example, the surface structure (39) may be trabecular metal.

The implant body (32) may have a diameter in the range 20-75 mm. The body (32) may have a diameter in the range 25-70 mm. The body (32) may have a diameter in the range 25-65 mm. Preferably, the diameter of the body (32) is in the range 35-65 mm.

The depth of the implant body (32) may be in the range 15-70 mm. The depth may be in the range 20-60 mm. Preferably, the depth is in the range 20-50 mm.

The thickness of the body (32) measured between the inner (33) and outer (34) surfaces may be in the range 0.75-20 mm.

The opening (38) of the body (32) may be circular or substantially circular. The circular opening (38) may have a diameter in the range 15-70 mm.

The implant body (32) may be made from plastic, metal or ceramic. Preferably, the implant body (32) is made from metal. The metal may be stainless steel. The metal may be titanium. The metal may be an alloy. The alloy may be cobalt-chrome alloy. The metal, metal alloy or ceramic could be coated or surface modified. The surface modification could be ceramic bonded to metal, oxinium, niobium nitride or titanium nitride, or carburisation.

The implant stem (35) may have a length in the range 10-200 mm. The stem (35) may have a diameter in the range 3-15 mm.

The implant stem (35) may be made from plastic, metal or ceramic. Preferably, the implant stem (35) is made from metal. The metal may be stainless steel. The metal may be titanium. The metal may be an alloy. The stem (35) may be integrally cast with the body (32).

FIG. 9 shows a cross-section of an implant (40) according to another embodiment of the present invention. The implant body (42) is based on a sphere with the outer surface (44) of the body (42) forming a shape that is more than hemispherical. The inner surface (43) comprises three substantially flat sections (41a,b,c). The implant (40) comprises a stem (45) having a first end (46) and a second end (47). The first end (46) of the stem (45) is fixed to the inner surface (43) of the pole of the implant body (42), such that the main axis of the stem (45) is coaxial with the opening (48) in the body (42). The second end (47) of the stem (45) extends through the opening (48) in the body (42).

The inner surface (43) of the implant body (42) comprises a surface structure (49) that enables bone in-growth. The surface structure (49) is shown as a generic feature for the purpose of clarity. The surface structure (49) is shown covering substantially all of the inner surface (43). However, the surface structure (49) may partially cover the inner surface (43), as described earlier in relation to FIGS. 1, 2, 4 and 5. The surface structure (49) may comprise projections as described herein (see for example FIGS. 11a-g). The surface structure (49) may comprise beads as described herein (see for example FIGS. 1 to 6). The surface structure (49) may mimic trabecular bone. For example, the surface structure (49) may be trabecular metal.

The implant body (42) may have a diameter in the range 20-75 mm. The body (42) may have a diameter in the range 25-70 mm. The body (42) may have a diameter in the range 25-65 mm. Preferably, the diameter of the body (42) is in the range 35-65 mm.

The depth of the implant body (42) may be in the range 15-70 mm. The depth may be in the range 20-60 mm. Preferably, the depth is in the range 20-50 mm.

The thickness of the body (42) measured between the inner (43) and outer (44) surfaces may be in the range 0.75-20 mm.

The opening (48) of the body (42) may be circular or substantially circular. The circular opening (48) may have a diameter in the range 15-70 mm.

The implant body (42) may be made from plastic, metal or ceramic. Preferably, the implant body (42) is made from metal. The metal may be stainless steel. The metal may be titanium. The metal may be an alloy. The alloy may be cobalt-chrome alloy. The metal, metal alloy or ceramic could be coated or surface modified. The surface modification could be ceramic bonded to metal, oxinium, niobium nitride or titanium nitride, or carburisation.

The implant stem (45) may have a length in the range 10-200 mm. The stem (45) may have a diameter in the range 3-15 mm.

The implant stem (45) may be made from plastic, metal or ceramic. Preferably, the implant stem (45) is made from metal. The metal may be stainless steel. The metal may be titanium. The metal may be an alloy. The stem (45) may be integrally cast with the body (42).

FIG. 10 shows a cross-section of an implant (50) according to another embodiment of the present invention. The implant body (52) is based on a sphere with the outer surface (54) of the body (52) forming a shape that is more than hemispherical. The inner surface (53) comprises four substantially flat sections (51a,b,c,d). The implant (50) comprises a stem (55) having a first end (56) and a second end (57). The first end (56) of the stem (55) is fixed to the inner surface (53) of the pole of the implant body (52), such that the main axis of the stem (55) is coaxial with the opening (58) in the body (52). The second end (57) of the stem (55) extends through the opening (58) in the body (52).

The inner surface (53) of the implant body (52) comprises a surface structure (59) that enables bone in-growth. The surface structure (59) is shown as a generic feature for the purpose of clarity. The surface structure (59) is shown covering substantially all of the inner surface (53). However, the surface structure (59) may partially cover the inner surface (53), as described earlier in relation to FIGS. 1, 2, 4 and 5. The surface structure (59) may comprise projections as described herein (see for example FIGS. 11a-g). The surface structure (59) may comprise beads as described herein (see for example FIGS. 1 to 6). The surface structure (59) may mimic trabecular bone. For example, the surface structure (59) may be trabecular metal.

The implant body (52) may have a diameter in the range 20-75 mm. The body (52) may have a diameter in the range 25-70 mm. The body (52) may have a diameter in the range 25-65 mm. Preferably, the diameter of the body (52) is in the range 35-65 mm.

The depth of the implant body (52) may be in the range 15-70 mm. The depth may be in the range 20-60 mm. Preferably, the depth is in the range 20-50 mm.

The thickness of the body (52) measured between the inner (53) and outer (54) surfaces may be in the range 0.75-20 mm.

The opening (58) of the body (52) may be circular or substantially circular. The circular opening (58) may have a diameter in the range 15-70 mm.

The implant body (52) may be made from plastic, metal or ceramic. Preferably, the implant body (52) is made from metal. The metal may be stainless steel. The metal may be titanium. The metal may be an alloy. The alloy may be cobalt-chrome alloy. The metal, metal alloy or ceramic could be coated or surface modified. The surface modification could be ceramic bonded to metal, oxinium, niobium nitride or titanium nitride, or carburisation.

The implant stem (55) may have a length in the range 10-200 mm. The stem (55) may have a diameter in the range 3-15 mm.

The implant stem (55) may be made from plastic, metal or ceramic. Preferably, the implant stem (55) is made from metal. The metal may be stainless steel. The metal may be titanium. The metal may be an alloy. The stem (55) may be integrally cast with the body (52).

FIGS. 11a-g show cross-sections of projections according to embodiments of the present invention. The projections enable bone to grow between, under and around them, thereby resulting in effective bone in-growth and a stable implant. Any shape or form of projection that allows such bone in-growth is in accordance with the present invention.

1. An implant comprising a hollow body having an opening, the body having an inner and an outer surface, wherein the inner surface of the body has a surface structure that enables bone in-growth.

2. An implant according to claim 1, wherein the inner surface is at least in part covered with projections.

3. An implant according to claim 2, wherein the majority of the inner surface is free of projections.

4. An implant according to claim 2, wherein the majority of the inner surface is covered with projections.

5. An implant according to claim 2, wherein substantially all of the inner surface is covered with projections.

6. An implant according to claim 1, wherein the inner surface is at least in part covered with recesses.

7. An implant according to claim 2, wherein the projections are at least in part in the form of beads.

8. An implant according to claim 1, wherein the body is at least in part curved.

9. An implant according to claim 8, wherein the inner surface is at least in part concave.

10. An implant according to claim 8, wherein the outer surface is at least in part convex.

11. An implant according to claim 8, wherein the body is part-spherical.

12. An implant according to claim 11, wherein the body is at least in part hemispherical.

13. An implant according to claim 11, wherein the body is more than hemispherical.

14. An implant according to claim 1, wherein the body encompasses 50-75% of a sphere.

15. An implant according to claim 1 further comprising a stem attached to the inner surface of the body.

16. An implant according to claim 15, wherein the stem extends through the opening in the body.

17. An implant according to claim 15, wherein the stem is fixedly attached to the body.

18. An implant according to claim 15, wherein the stem is removably attached to the body.

19. An implant according to claim 18, wherein the body and stem have corresponding threads.

20. An implant according to claim 15, wherein the main axis of the stem is co-linear with the centre of the opening in the body.

21. An implant according to claim 1, wherein the implant is a femoral implant.

22. An implant according to claim 1, wherein the surface structure comprises a coating.

23. An implant according to claim 2, wherein the projections comprise a coating.

24. An implant according to claim 22, wherein the coating stimulates bone in-growth.

25. An implant according to claim 22, wherein the coating comprises hydroxyapatite.

26. An implant according to claim 15, wherein the stem is made from a material selected from the group consisting of plastic, metal, ceramic and resorbable materials.

27. (canceled)

28. A method of implantation, comprising:
preparing a resected bone with an optional cavity in the bone canal;
providing an implant according to claim 1;
implanting the implant.

29. A method according to claim 28, wherein the bone is a femur.

30. (canceled)
